SEQUENCE LISTING (14 sequences)

SEQ ID NO: 1 - DNA, 2672 bp, Caenorhabditis elegans
ggtttaattacccaagtttgaggttcacaatgattcgtgtttatttgataattttaatgcatttggtgattggtttaacccagaacaattcaactacaccttcgccaattatcacctcaagtaacagctctgtacttgtattcgagatttcttcaaaaatgaaaatgatcgaaaaaaagctggaagccaacacagtccatgtccttcgcctggaattagatcaaagtttcatattagatttaaccaaagtcgccgcggaaatcgttgattcttcgaaatacagtaaagaagacggtgttatactcgaagtaacagtttcaaatggccgtgatagttttttattgaaacttccgacggtttatccgaacttgaagctctatactgacggaaaactgctcaatccgctcgttgagcaagatttcggggcgcacagaaagaggcacaggataggcgaccctcatttccatcaaaacctgatcgtaaccgtgcagtctcgattgaatgctgatatagattataggcttcatgtgactcatttggatcgggcccaatatgattttctgaagttcaagacgggacagaccacgaaaacgttgtcgaatcagaagctgacgtttgtcaagccgattggattttttttgaattgcagcgaacaaaatatttcccaattccacgtcacattgtacagtgaagatgatatttgtgcaaatctgataactgtgccggcgaatgaatccatctatgatcgatcagtgatttccgataaaactcacaatcgacgtgtcctatcattcaccaaaagagccgacatttttttcactgaaactgaaatctcgatgttcaaatcattccgaatcttcgtcttcatagctcccgatgattctggatgttctaccaacacatcacgcaaaagtttcaacgagaaaaagaaaatatcttttgaattcaaaaaactggaaaatcaatcatacgccgtcccgacggctttgatgatgatatttctgacgacaccgtgtcttttgttccttccaattgtgattaatattatcaagaatagcagaaaattggcaccatcacaatcaaatcttatctcattttctccagttccgtctgagcaacgggacatggatttgagccatgatgagcagcagaatacgagctcagaactcgaaaataatggagaaattccagcagcagaaaatcaaattgttgaagagatcacggctgaaaatcaagaaacgagcgtagaagagggaaaccgggaaattcaagttaaaattccgttgaaacaggattcattatcactccatggccaaatgcttcaatatcccgttgcaataattctcccagttctcatgcacacagctatcgaattccataaatggacgacatctacaatggcaaatcgcgacgaaatgtgcttccacaatcatgcgtgtgctcggccattgggagaacttcgagcttggaataatatcatcaccaatataggatatactctttatggagccatcttcattgttttgtcgatatgtagaagaggccgtcatgagtattctcatgtttttggtacatatgaatgcacacttttagatgtgactattggtgttttcatggttttgcaatcaattgctagtgccacttatcatatttgccccagtgatgtggcttttcagtttgatacgccgtgcatccaagttatctgtggacttctcatggtccgtcagtggtttgttcgtcacgaatctccatcaccagcctatacaaatatcctactagttggagttgtctccttgaactttctaatatctgcattctccaaaacatcatatgtccgattcatcatcgctgtaattcatgtcattgtcgttggatcgatctgtttggcaaaggaaagatccttgggatcggaaaaattaaaaactcgatttttcatcatggccttctcgatgggaaatttcgcagcaatcgtgatgtatctgacgctttcggcgtttcatttgaatcaaatagccacgtattgctttattataaattgtatcatgtatctgatgtactatggatgcatgaaagttttacatagcgagagaataacgtcgaaggctaaactttgtggagctctgtcactgctcgcgtgggctgttgccggatttttcttctttcaagatgatacagattggacgagatctgcggcggcgagccgagcactcaacaagccatgcctgctactcggcttcttcggttcccacgatttatggcacatcttcggagcattggccggtcttttcacattcattttcgtctcctttgttgatgatgatctcattaatacacgcaaaacttcgattaacattttctagagtaattttgttttccctatccggtttttcatatttcaactttttctcctgccttaatacgtagcccgatctctcatttcttcatgttttaagaactttctgaatctatgtaattagttggtctactttgaaacatttttactgtattcatctccacggctccctacctccctgtgaatataattccaaatttagctcctctctacgcagtgatctggtgtttataacatgtttatttatttttcccaacatttcccaatttcaggtgttcacttgtttgtttgctccccctctatgcataaattttcagcc

SEQ ID NO: 2 - protein, 776 aa, Caenorhabditis elegans
MIRVYLIILMHLVIGLTQNNSTTPSPIITSSNSSVLVFEISSKMKMIEKKLEANTVHVLRLELDQSFILDLTKVAAEIVDSSKYSKEDGVILEVTVSNGRDSFLLKLPTVYPNLKLYTDGKLLNPLVEQDFGAHRKRHRIGDPHFHQNLIVTVQSRLNADIDYRLHVTHLDRAQYDFLKFKTGQTTKTLSNQKLTFVKPIGFFLNCSEQNISQFHVTLYSEDDICANLITVPANESIYDRSVISDKTHNRRVLSFTKRADIFFTETEISMFKSFRIFVFIAPDDSGCSTNTSRKSFNEKKKISFEFKKLENQSYAVPTALMMIFLTTPCLLFLPIVINIIKNSRKLAPSQSNLISFSPVPSEQRDMDLSHDEQQNTSSELENNGEIPAAENQIVEEITAENQETSVEEGNREIQVKIPLKQDSLSLHGQMLQYPVAIILPVLMHTAIEFHKWTTSTMANRDEMCFHNHACARPLGELRAWNNIITNIGYTLYGAIFIVLSICRRGRHEYSHVFGTYECTLLDVTIGVFMVLQSIASATYHICPSDVAFQFDTPCIQVICGLLMVRQWFVRHESPSPAYTNILLVGVVSLNFLISAFSKTSYVRFIIAVIHVIVVGSICLAKERSLGSEKLKTRFFIMAFSMGNFAAIVMYLTLSAFHLNQIATYCFIINCIMYLMYYGCMKVLHSERITSKAKLCGALSLLAWAVAGFFFFQDDTDWTRSAAASRALNKPCLLLGFFGSHDLWHIFGALAGLFTFIFVSFVDDDLINTRKTSINIF

SEQ ID NO: 3 - DNA, 20 bp, Artificial Sequence
  primer
ggtcatgagagggtcgagag

SEQ ID NO: 4 - DNA, 37 bp, Artificial Sequence
  primer
aacgcctagggaaaatgttaatcgaagttttgcgtgt

SEQ ID NO: 5 - DNA, 37 bp, Artificial Sequence
  primer
ctaagctagcatgagtaaaggagaagaacttttcact

SEQ ID NO: 6 - DNA, 19 bp, Artificial Sequence
  primer
tcaccgtcatcaccgaaac

SEQ ID NO: 7 - DNA, 31 bp, Artificial Sequence
  primer
aaaaactgcagggtcatgagagggtcgagag

SEQ ID NO: 8 - DNA, 34 bp, Artificial Sequence
  primer
acgcggatccggaaaaatgaggagttttaatttc

SEQ ID NO: 9 - DNA, 22 bp, Artificial Sequence
  primer
tggatacgctaacaacttggaa

SEQ ID NO: 10 - DNA, 20 bp, Artificial Sequence
  primer
gcggaaatcgttgattcttc

SEQ ID NO: 11 - DNA, 21 bp, Artificial Sequence
  primer
gtttaattacccaagtttgag

SEQ ID NO: 12 - DNA, 20 bp, Artificial Sequence
  primer
gtgccataaatcgtgggaac

SEQ ID NO: 13 - DNA, 20 bp, Artificial Sequence
  primer
ggtcatgagagggtcgagag

SEQ ID NO: 14 - DNA, 21 bp, Artificial Sequence
  primer
gcaaacgagcaaattgtgaag

(19) United States
(12) Patent Application Publication
(10) Pub. No.: US 2009/0077679 A1
(43) Pub. Date: Mar. 19, 2009

(54) GENE SILENCING BY SYSTEMIC RNA INTERFERENCE

(75) Inventors: Craig P. Hunter, Arlington, MA (US); William M. Winston, Somerville, MA (US); Christina Molodowitch, Somerville, MA (US)

Correspondence Address: BURNS & LEVINSON, LLP, 125 SUMMER STREET, BOSTON, MA, 02110, US

(73) Assignee: PRESIDENT AND FELLOWS OF HARVARD COLLEGE, Quincy, MA (US)

(21) Appl. No.: 12/035,979

(22) Filed: Feb. 22, 2008

Related U.S. Application Data
(63) Continuation of application No. 10/304,930, filed on Nov. 26, 2002, now abandoned.
(60) Provisional application No. 60/333,325, filed on Nov. 26, 2001.

(51) Int. Cl. A01K 67/00; C07H 21/04; G01N 33/53; C07K 14/00; C12N 15/09; C07K 16/18; C12N 15/64; C12N 5/10

(52) U.S. Cl. 800/16; 536/23.1; 436/94; 435/320.1; 435/348; 800/13; 435/366; 800/17; 800/18; 800/14; 530/350; 530/387.1; 530/388.1; 436/86; 435/455

(57) ABSTRACT

Nucleic acid and protein sequences relating to a gene required for systemic RNAi are disclosed. The SID-1 protein is shown to be required for systemic RNAi. Nucleic acids, vectors, transformed cells, transgenic animals, polypeptides, and antibodies relating to the sid-1 gene and protein are disclosed. Also provided are methods for reducing the expression of a target gene in a cell, a population of cells, or an animal.

### RELATED APPLICATIONS

This application claims the benefit of U.S. Provisional Application Ser. No. 60/333,325, filed on Nov. 26, 2001, the entire contents of which are incorporated herein.

### GOVERNMENT SUPPORT

This invention was sponsored by NSF grant MCB-0110452 and the government has certain rights to the invention.

### FIELD OF THE INVENTION

This invention relates to genes and proteins involved in systemic RNA interference.

### BACKGROUND OF THE INVENTION

Double-stranded RNA-mediated gene interference (RNAi) is a mechanism of gene silencing observed in a variety of organisms, including nematodes, insects, and mammals (see, e.g., Carthew (2001) Current Opinion in Cell Biology 13:244-248; Zamore (2001) Nature Structural Biology 8:746-750; Hunter (1999) Current Biology 9:R440-R442). A related phenomenon, post-transcriptional gene silencing (PTGS) has been observed in plants (Vaucheret (2001) J Cell Science 114:3083-3091). RNAi and PTGS occur when the presence of a double-stranded RNA molecule in an organism reduces or silences expression of a gene with a common sequence. RNAi has therefore been exploited extensively in experimental model animals to selectively inactivate particular genes.

The mechanism by which RNAi inactivates target genes has been explored. Introduction of a double-stranded RNA substrate is associated with the appearance of 21-26 nucleotide small-interfering RNAs (siRNAs) believed to mediate RNAi in C. elegans and Drosophila. The generation of siRNAs has been attributed to an RNAse III enzyme called Dicer that is also implicated in the processing of small temporal RNAs in C. elegans and similar small RNAs in human cells. Synthetic siRNAs can trigger RNAi in C. elegans, Drosophila, and cultured mammalian cells.

Genetic analyses have identified a number of genes that are required for RNAi and related phenomena in C. elegans, Neurospora crassa, and Arabidopsis thaliana. In C. elegans, two major classes of RNAi defective (rde) mutants have been described. Genes of the first class are involved not only in RNAi, but in other processes as well, as mutants of this class display phenotypes such as chromosome non-disjunction, temperature-sensitive sterility, and increases in germline transposon mobility in addition to defects in RNAi. Genes of the second class are those in which the only readily detectable phenotype is resistance to RNAi, and which therefore appear to be specifically involved in RNAi. Genetic analyses indicate that genes of this class are involved in the initiation of RNAi. Homologs of the rde genes which appear to be specific to RNAi have been isolated from Arabidopsis thaliana and Neurospora crassa.

A notable aspect of RNAi in C. elegans and of PTGS in plants is that silencing can spread throughout the organism and be passed on from parent to progeny. This phenomenon, known as systemic RNAi, does not appear to act stoichiometrically. For example, injecting a wild-type adult nematode with an estimated 60,000 double-stranded unc-22 RNA molecules produces at least 100 strongly affected progeny. Each of these progeny has 550 cells at hatching, meaning that the injected double-stranded RNA is diluted to less than two molecules per cell. Thus, a mechanism for perpetuating silencing must be employed by the organism. It has been suggested that the RNA is acting catalytically and/or is replicated by cellular proteins. However, the mechanism of systemic RNAi and genes involved in mediating systemic RNAi have not been identified to date.

The inventors have identified genes that are required for systemic RNAi. The inventors have also identified an additional C. elegans gene with significant sequence homology. Genes involved in systemic RNAi can be used to investigate the mechanism of RNAi, and to modulate gene expression in a variety of organisms, including C. elegans, plants, mice, humans, and others.

### SUMMARY OF THE INVENTION

The present invention provides sid-1 genes, polypeptides encoded by sid-1 genes, and methods for using sid-1 genes to silence gene expression or to transmit gene silencing in population of cells or in an animal.

In one aspect, the present invention provides isolated nucleic acids corresponding to all or part of a sid-1 gene. In some embodiments, the isolated nucleic acids include a nucleotide sequence of at least 10, 12, 14, 16, or 18 consecutive nucleotides of SEQ ID NO:1, or a sequence complementary thereto. In other embodiments, the nucleic acids include nucleotide sequences encoding a SID-1 protein, at least a transmembrane domain of a SID-1 protein, at least an extracellular domain of a SID-1 protein, or at least a serpin domain of a SID-1 protein. In particular embodiments, the nucleic acids include a sequence of SEQ ID NO:1, a sequence encoding a polypeptide comprising amino acid residues 19 to 314] of SEQ ID NO:2, and a sequence encoding a polypeptide comprising residues 314-339, 425-451, 481-502, 509-541, 546-571, 575-599, 601-621, 633-655, 659-681, 692-712, or 742-766 of SEQ ID NO:2.

In another aspect, the invention provides isolated nucleic acids encoding polypeptides having at least 80%, at least 85%, at least 90%, or 95% amino acid sequence identity with a SID-1 protein; at least a transmembrane domain of a SID-1 protein; or at least an extracellular domain of a SID-1 protein. In some embodiments, the isolated nucleic acids encode a polypeptide having at least 80%, 85%, 90%, or 95% amino acid sequence identity with a SID-1 protein and having SID-1 activity in a cell capable of expressing SID-1 activity.

In another aspect, the invention provides isolated nucleic acids that hybridize to at least a portion of a nucleic acid of SEQ ID NO: 1 under conditions including a wash step of 1.0×SSC, a wash step of 0.5×SSC, a wash step of 0.2×SSC, or a wash step of 0.1×SSC. In some embodiments, the isolated nucleic acids encode a polypeptide having SID-1 activity.

In another aspect, the invention provides a nucleic acid comprising a nucleotide sequence encoding a polypeptide having SID-1 activity, and that hybridizes to at least a portion of a nucleic acid of SEQ ID NO:1 under conditions including a wash step of 1.0×SSC at 65° C., a wash step of 0.5×SSC, a wash step of 0.2×SSC, or a wash step of 0.1×SSC, and that is operably joined to a heterologous regulatory region such that the sequence is expressed.

In another aspect, the invention provides a kit for detecting at least a portion of a sid-1 nucleic acid. The kits can include any of the foregoing isolated nucleic acids of the invention, and a means for detecting the isolated nucleic acid. In some embodiments, the means for detecting the isolated nucleic acid includes a detectable label bound thereto, and, in some embodiments, the means includes a labeled secondary nucleic acid, which specifically hybridizes to the first isolated nucleic acid.

In another aspect, the invention provides a vector including any of the foregoing isolated nucleic acids of the invention. In some embodiments, the vector includes a genetic construct capable of expressing the nucleic acids of the invention. In some embodiments, the nucleic acids of the invention are operably joined to a heterologous regulatory region and, in some embodiments, the nucleic acids are operably joined to heterologous coding sequences to form a fusion vector. In some embodiments, the vector includes a SID-1 regulatory region and, in some embodiments, the SID-1 regulatory region is operably joined to a heterologous coding sequence.

In another aspect, the invention provides cells transformed with the foregoing nucleic acids of the invention, or a genetic construct capable of expressing a nucleic acid of the invention. In some embodiments, the nucleic acid of the invention is operably joined to heterologous coding sequences to encode a fusion protein. In some embodiments, the cells are bacterial cells, yeast cells, insect cells, nematode cells, amphibian cells, rodent cells, or human cells. In some embodiments, the cells are mammalian somatic cells, fetal cells, embryonic stem cells, zygotes, gametes, germ line cells, and transgenic animal cells.

In another aspect, the invention provides non-human transgenic animals. In these aspects, a genetic construct has introduced a modification into a genome of the animal, or an ancestor of the animal, and the modification includes insertion of a nucleic acid encoding at least a fragment of a SID-1 protein, at least a transmembrane portion of a SID-1 protein, or at least an extracellular domain of a SID-1 protein. In some embodiments, the animals are rats, nice, hamsters, guinea pigs, rabbits, dogs, cats, goats, sheep, pigs, and non-human animals.

In another aspect, the invention provides substantially pure protein preparations including polypeptides selected from a SID-1 protein; at least a transmembrane domain of a SID-1 protein; and at least an extracellular domain of a SID-1 protein. In particular embodiments, the peptide is selected from amino acids 19-314, 425-451, 481-502, 509-541, 546-571, 575-599, 601-621, 633-655, 659-681, 692-712, and 742-766 of SEQ ID NO:2.

In another aspect, the invention provides a substantially pure protein preparation including polypeptides having at least 80%, 85%, 90%, or 95% amino acid sequence identity with a SID-1 protein; at least a transmembrane domain of a SID-1 protein; or at least an extracellular domain of a SID-1 protein. In some embodiments, the substantially pure preparation includes a polypeptide having at least 80%, 85%, 90%, or 95% amino acid sequence identity with a SID-1 protein and having SID-1 activity in a cell capable of expressing SID-1 activity.

In another aspect, the invention provides a substantially pure antibody preparation including an antibody raised against a SID-1 polypeptide. In some embodiments, the antibody is a monoclonal antibody. In some embodiments, the antibody is an Fab fragment, an F(ab)′2 fragment, an Fv fragment, or a single-chain Fv fragment (ScFv).

In another aspect, the invention provides a kit for detecting at least an epitope of a SID-1 protein. The kits include an anti-SID-1 antibody of the invention and a means for detecting said antibody. In some embodiments, the means for detecting said anti-SID-1 antibody includes a detectable label bound thereto and, in some embodiments, the means for detecting said anti-SID-antibody includes a labeled secondary antibody which specifically binds to the anti-SID-1 antibody.

In another aspect, the invention provides a method for reducing the expression of a target gene in a cell comprising the steps of introducing a nucleic acid vector comprising a sid-1 sequence into the cell and introducing a double-stranded RNA molecule having a sequence complementary to the target gene, wherein the sid-1 nucleic acid sequence encodes a polypeptide having SID-1 activity.

In another aspect, the invention provides a method for reducing the expression of a target gene in a population of cells, comprising the steps of introducing a nucleic acid vector comprising a sid-1 nucleic acid sequence into at least a portion of the population of cells and introducing a double-stranded RNA molecule having a sequence complementary to the target gene, wherein the sid-1 nucleic acid sequence encodes a polypeptide having SID-1 activity.

In another aspect, the invention provides a method for reducing the expression of a target gene in an animal, the method comprising introducing a nucleic acid vector comprising a sid-1 sequence into the animal, and introducing a double-stranded RNA molecule having a sequence complementary to the target gene, wherein the sid-1 sequence encodes a polypeptide having SID-1 activity.

### BRIEF DESCRIPTION OF THE DRAWING

FIG. 1 is a graphic representation of the levels of embryonic lethality in sid-1 hermaphrodites injected with mex-3 RNA.

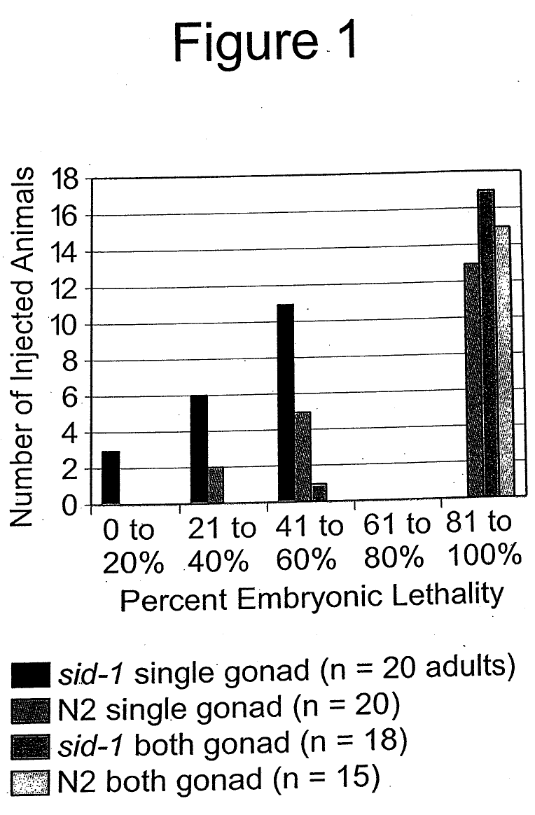

FIG. 2 is a diagrammatic representation of the structures of the SID-1 genomic locus and the SID-1 polypeptide.

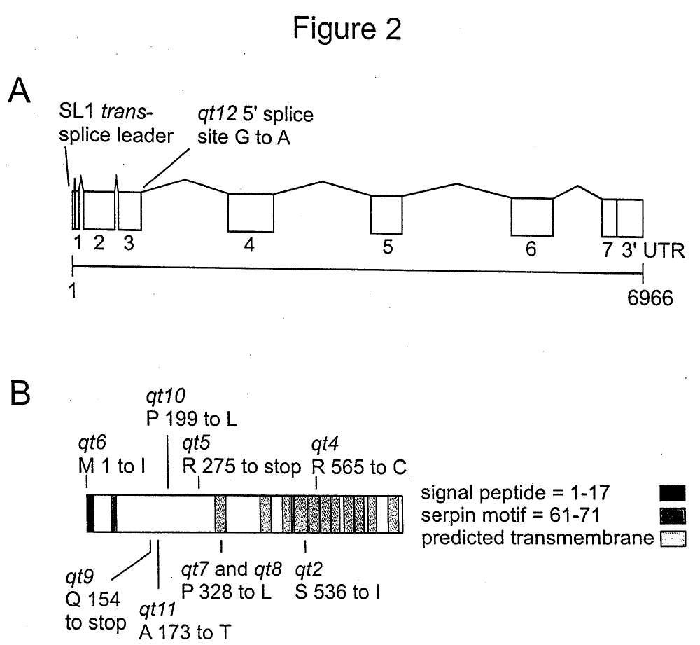

FIG. 3 is a diagrammatic representation of the nucleotide sequence of the sid-1 sequence.

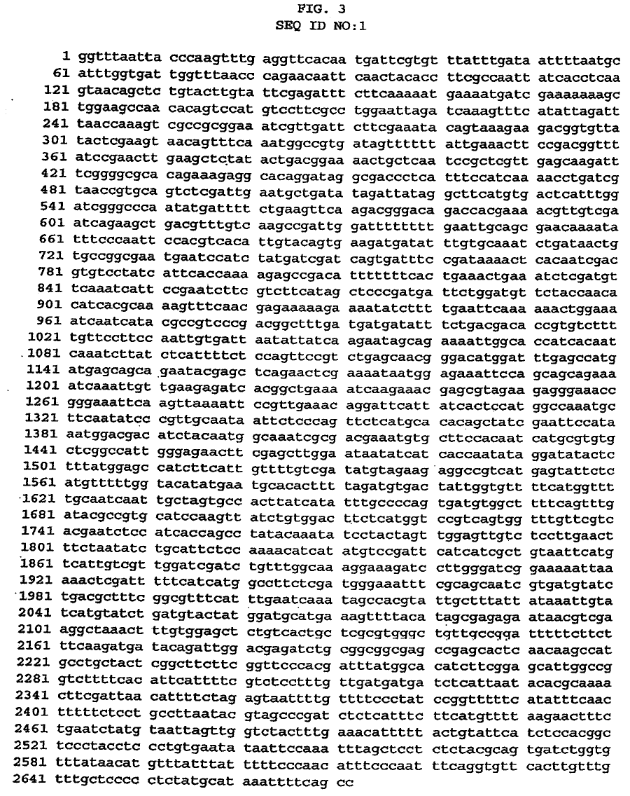

FIG. 4 is a diagrammatic representation of the amino acid sequence of the SID-1 polypeptide.

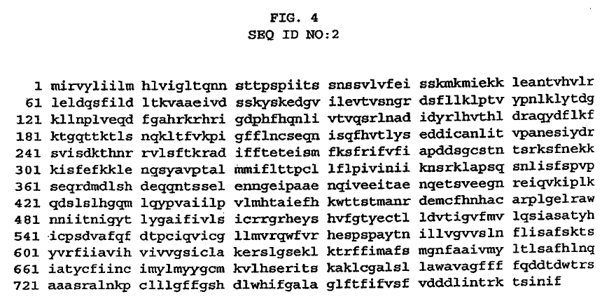

### DETAILED DESCRIPTION OF THE INVENTION

The patents, published applications, and scientific publications referred to herein establish knowledge that was available to those of ordinary skill in the art at the time the invention was made. The entire disclosures of the issued U.S. patents, published and pending patent applications, and other references cited herein are hereby incorporated by reference.

The present invention depends, in part, upon the identification, isolation, and characterization of a gene encoding a transmembrane protein that plays a significant role in propagating gene silencing by RNAi from one cell to another and from parent to progeny. The gene has been designated sid-1 to indicate that cells and animals lacking the gene or the protein are systemic RNA interference deficient. Elimination of sid-1 function in an animal inhibits the spreading of RNAi from one cell to another and from parent to progeny. SID-1 is found in organisms where systemic RNA interference is observed, such as C. elegans, but not in organisms where systemic RNA interference is not found, such as Drosophila melanogaster. Elimination of sid-1 in isolated cells in culture also inhibits RNAi in cells exposed to a double-stranded (dsRNA) in the culture medium. Thus, SID-1 is involved in importing and/or processing the systemic RNAi signal across a cell membrane.

The sid-1 gene encodes a 776 amino acid protein with eleven transmembrane domains as determined by analysis using the TMPRED, SOSUI, and TMHMM2.0 programs, an extracellular domain, a serpin motif, and a signal peptide, consistent with a molecule that acts to transmit a signal and/or transport molecules across a cell membrane.

In one aspect, the present invention provides nucleic acid molecules, or nucleic acid analogs, having sid-1 sequences, or useful fragments thereof. The full length cDNA of the C. elegans sid-1 gene is disclosed as SEQ ID NO:1 and as GenBank Accession No. AF478687.

Nucleic acid molecules of the invention may be DNA or RNA molecules, or hybrid DNA-RNA molecules. As used herein, a “nucleic acid analog” means a molecule having sufficient structural and functional similarity to a nucleic acid to direct sequence-specific forward or reverse transcription of complementary nucleic acids, or to direct sequence-specific translation of an encoded polypeptide within a living cell. The nucleic acid analogs of the invention may be any of those known in the art, such as peptide nucleic acids, analogs including modified bases (e.g., 2′-halogeno-2′-dexynucleosides) and/or analogs including modified internucleoside linkages (e.g., phosphorothioate linkages), which are useful in applications such as in vitro translation or antisense technologies. In the remainder of this disclosure and the appended claims, whenever the term “nucleic acids” is used, the term is intended to embrace nucleic acid analogs when such analogs would be useful or suitable in the context of the usage. The nucleic acids may be sense molecules corresponding to all or a portion of a sid-1 gene sequence, or may be antisense molecules that are complementary to all or a portion of a sid-1 gene sequence. The nucleic acids may be derived from or correspond to genomic DNA or cDNA, or may be synthetic molecules based upon a sid-1 protein sequence and the genetic code (e.g., synthetic nucleic acids which reflect the codon usage preferences in the host cells used in an expression system).

In some embodiments, the sid-1 nucleic acids comprise the entire coding region of a sid-1 gene (e.g., SEQ ID NO:1). Such nucleic acids can be used to produce genetic constructs for transformation of cells, or for in vitro transcription and translation systems. Such nucleic acids can also be used as probes in hybridization assays to detect sid-1 sequences in samples of other nucleic acids.

In other embodiments, subsets of the SID-1 nucleic acid sequences are provided for use as primers for nucleic acid amplification reactions, as probes in hybridization assays to detect SID-1 sequences in samples of other nucleic acids, or as probes to distinguish normal or wild-type sequence from abnormal or mutant sequences. In these embodiments, the nucleic acids of the invention comprise at 10, preferably at least 12, more preferably at least 14, more preferably at least 16, and most preferably at least 18 consecutive nucleotides selected from a SID-1 sequence such as SEQ ID NO:1. Depending upon the nature of the application, it may be preferable to choose SID-1 sequences which will have unique targets, or which are expected to have unique targets, within a sample being probed or amplified. Thus, for example, sequences that are longer and sequences that do not include frequently repeated elements (for example, polyadenylation signals) are more likely to be uniquely represented within any given sample. For purposes of choosing primers for amplification reactions, sequences of at least 15, and preferably 18-25 nucleotides are preferred.

In certain preferred embodiments, nucleic acids are provided which encode structural domains of a SID-1 protein, or which encode fragments of the protein that may serve as epitopes for the generation of antibodies. Thus, for example, preferred nucleic acids include those encoding the transmembrane domains of the SID-1 proteins (i.e., approximately residues 425-451, 481-502, 509-541, 546-571, 575-599, 601-621, 633-655, 659-681, 692-712, and 742-766 of SEQ ID NO:2. and allelic variants and homologs thereof), or encoding the extracellular domain (i.e., approximately residues 19-314 and allelic variants and homologs thereof). Other preferred nucleic acid acids include those encoding epitopes of the SID-1 proteins having high predicted antigenicity, as identified by standard sequence analysis techniques described below.

In certain embodiments, nucleic acids are provided which encode polypeptides having at least 80%, and preferably at least 85%, 90% or 95% amino acid sequence identity with at least a structural domain of a SID-1 protein. As used herein with respect to nucleic acid and amino acid sequences, the term “identity” means a measure of the degree of similarity of two sequences based upon an alignment of the sequences which maximizes identity and which is a function of the number of identical nucleotides or residues, the number of total nucleotides or residues, and the presence and length of gaps in the sequence alignment. A variety of algorithms and computer programs are available for determining sequence identity using standard parameters. For example, Gapped BLAST or PSI-BLAST (Altschul et al. (1997) Nucleic Acids Res. 25:33 89-3402), BLAST (Altschul et at. (1990) J. Mol. Biol. 215:403-410), and Smith-Waterman (Smith et al. (1981) J. Mol. Biol. 147:195-197). As used herein, percent identity is based upon the default values for the BLAST algorithms.

Thus, in some embodiments, a nucleic acid is provided which encodes a polypeptide having at least 80%, 85%, 90% or 95% amino acid sequence identity with a transmembrane domain of a SID-1 proteins (e.g., approximately residues 425-451, 481-502, 509-541, 546-571, 575-599, 601-621, 633-655, 659-681, 692-712, and 742-766 of SEQ ID NO:2, and allelic variants and homologs thereof), an extracellular domain (e.g., approximately residues 19-314 and allelic variants and homologs thereof). In some preferred embodiments, nucleic acids are provided encoding a polypeptide having at least 80%, 85%, 90% or 95% amino acid sequence identity with a SID-1 protein and having SID-1 activity. The ability of a protein to exhibit SID-1 activity can be measured by its ability to complement a SID-1 −/− mutant (e.g., a SID-1 knock-out mutant) and restore a normal or SID-1 +/+ phenotype (e.g., to restore systemic RNA interference) in a cell otherwise capable of expressing SID-1 activity (e.g., a dissociated embryonic cell from the SID-1 −/− mutant), or confer a SID-1 phenotype on a cell otherwise lacking SID-1 activity.

As used herein, the term “mutation” refers to a change in a nucleic acid sequence, whether or not expressed as a change in a corresponding encoded protein sequence, relative to some reference sequence. The reference sequence may be a “wild-type” sequence (i.e., one or more high frequency sequences in a population corresponding to a “normal” phenotype), or any other sequence. As used herein, the term mutation is intended to be synonymous with the term polymorphism, and therefore the differences between any two non-identical sequences may be regarding as mutations. The term mutation is intended to encompass insertions, deletions and/or substitutions of one or more nucleotides relative to a reference sequence. Thus, in some embodiments, the invention provides nucleic acids encoding sid-1 sequence that contain polymorphisms when compared to the sequence of, for example, SEQ ID NO:1.

In other embodiments, isolated nucleic acids are provided which include a nucleotide sequence that hybridizes to at least a portion of a sid-1 coding sequence (e.g., SEQ ID NO: 1) under stringent hybridization conditions. Such conditions include hybridizations employing a wash step of 1.0×SSC at 65° C., and equivalents thereof. More stringent conditions can include wash steps of 0.5×SSC, 0.2×SSC, or even 0.1×SSC. Other equivalently stringent conditions are well known in the art. See, e.g., Ausubel et al., eds. (1989) Current Protocols in Molecular Biology, Vol. I, John Wiley & Sons, Inc., New York, and Sambrook et al (1989) Molecular Cloning: A Laboratory Manual, Cold Spring Harbor Laboratories, New York; and Davis et al. (1986). In preferred embodiments, the nucleic acid encodes a polypeptide having SID-1 activity.

In another aspect, the invention provides nucleic acids, either isolated or existing within cells, in which a nucleotide sequence encoding a polypeptide having SID-1 activity is operably joined to a heterologous regulatory region such that the SID-1 polypeptide is expressed. As used herein, the terms “exogenous” or “heterologous” mean, with respect to two or more genetic sequences, that the genetic sequences do not occur in the same physical relation to each other in nature and/or do not naturally occur within the same genome. For example, a genetic construct may include a coding region which is operably joined to one or more regulatory elements, and these sequences are considered heterologous to each other if they are not operably joined in nature and/or they are not found in the same genome in nature. Similarly, a genetic construct which is introduced into a cell is considered heterologous to that cell to the extent that it contains genetic sequences not found in that cell. In addition, a synthetically-produced genetic sequence based upon a naturally occurring sequence, will be heterologous to the naturally-occurring sequence to the extent codons have been altered and the synthetic sequence does not exist in nature. Allelic variants of a sequence in a species are not considered heterologous to each other.

As used herein, the term “operably joined” refers to a covalent and functional linkage of genetic regulatory elements and a genetic coding region which can cause the coding region to be transcribed into mRNA by an RNA polymerase which can bind to one or more of the regulatory elements. Thus, a regulatory region, including regulatory elements, is operably joined to a coding region when RNA polymerase is capable under permissive conditions of binding to a promoter within the regulatory region and causing transcription of the coding region into mRNA. In this context, permissive conditions would include standard intracellular conditions for constitutive promoters, standard conditions and the absence of a repressor or the presence of an inducer for repressible/inducible promoters, and appropriate in vitro conditions, as known in the art, for in vitro transcription systems.

Thus, in certain embodiments, a heterologous regulatory region may be inserted into a chromosome such that it is operably joined to an endogenous SID-1 sequence. In some embodiments, the polypeptide has at least 80%, 85%, 90% or 95% amino acid sequence identity with an amino acid sequence of SEQ ID NO: 2. In other embodiments, the nucleic acid encoding the polypeptide hybridizes to at least a portion of a nucleic acid of SEQ ID NO: 1 under conditions including a wash step of 1.0×SSC at 65° C., 0.5×SSC, 0.2×SSC, or 0.1×SSC.

In certain embodiments, the nucleic acids of the invention encode polypeptides including a SID-1 sequence of at least 50 amino acid residues in length, and preferably at least 100, 200 or 300 amino acid residues in length. These polypeptides can include a SID-1 sequence which includes at least one transmembrane domain, or at least one extracellular domain. In some preferred embodiments, the polypeptide has SID-1 activity. Such activity may be the retention and or transport of interfering RNA molecules into a cell, or the restoration or initiation of systemic RNA interference in an organism lacking systemic RNA interference.

In another aspect, the invention provides kits for detecting at least a portion of a sid-1 nucleic acid (i.e., sid-1 genomic DNA, mRNA, cDNA or amplification products thereof). The kits include an isolated nucleic acid of the invention as a probe and means for detecting the probe. The means for detecting the probe can be a detectable label bound to the probe or a secondary nucleic acid probe for detecting the first probe (e.g., labeled secondary nucleic acid which specifically hybridizes to the isolated nucleic acid.).

In another aspect, the present invention provides genetic constructs comprising sequences selected from sid-1 genes. As used herein, the phrase “genetic construct encoding a SID-1 protein” means a recombinant DNA, RNA, or nucleic acid analog molecule which includes a genetic sequence encoding, or which is complementary to a genetic sequence encoding, the amino acid sequence of the SID-1 protein, and which is capable of being expressed in a cell which has been transformed with the construct. The construct may express the SID-1 protein transiently, or may stably integrate into the genome of the cell and express the protein conditionally or constitutively.

In one series of embodiments, sid-1 coding sequences (e.g., the entire coding region, sequences encoding structural domains, sequences encoding potential epitopes, or sequences encoding useful primers or probes) are operably joined to an endogenous or exogenous regulatory region to form an expression construct. Useful regulatory regions for these purposes include the endogenous SID-1 regulatory region, constitutive promoter sequences (e.g., CMV, SV40, EF2), inducible promoter sequences (e.g., lacZ, tet).

As used herein, the term “vector” means any genetic construct, such as a plasmid, phage, transposon, cosmid, chromosome, virus, virion, etc., which is capable transferring gene sequences between cells. Vectors may be capable of one or more of replication, expression, and insertion or integration, but need not possess each of these capabilities. Thus, the term includes cloning, expression, homologous recombination, and knock-out vectors.

Many useful vector systems are now widely available. For example, useful bacterial vectors include, but are not limited to, pQE70, pQE60, pQE-9 (Qiagen, Valencia, Calif.), pBluescript II (Stratagene, La Jolla, Calif.), and pTRC99a, pKK223-3, pDR540 and pRIT2T (Pharmacia, Piscataway, N.J.), pTrc (Amann et al. (1988) Gene 69:301-315) and pET 11d (Studier et al. (1990) Methods in Enzymol. 185:60-89). A wide variety of vectors can be used to transform nematodes, as DNA is readily replicated and transmitted in nematodes such as C. elegans (see, e.g. Mello C. et al. (1995) Methods Cell Biol. 48:451-82).

Examples of vectors for expression in yeast include pYepSec1 (Baldari et al. (1987) EMBO J. 6:229-234), pMFa (Kurjan et al. (1982) Cell 30:933-943), pJRY88 (Schultz et al. (1987) Gene 54:113-123), and pYES2 (Invitrogen Corporation, San Diego, Calif.). The SID-1 proteins can also be expressed in insect cells (e.g., Sf 9 cells) using, for example, baculovirus expression vectors including, but not limited to, pAc vectors (Smith et al. (1983) Mol Cell Biol. 3:2156-2165) and pVL vectors (Lucklow et al. (1989) Virology 170:31-39). Examples of mammalian expression vectors include, but are not limited to, pCDM8 (Seed (1987) Nature 329:840) and pMT2PC (Kaufman et al. (1987) EMBO J. 6:187-195). Other useful eukaryotic vectors include, but are not limited to, pXT1, pSGS (Stratagene, La Jolla, Calif.), and pSVK3, pBPV, pMSG, and PSVLSV40 (Pharmacia, Piscataway, N.J.). Thus, one of ordinary skill in the art can choose a vector system appropriate to the host cell to be transformed.

In other embodiments, the vectors comprise defective or partial SID-1 sequences in a “knock-out” vector. Such vectors are well-known in the art and can be used to produce a transgenic organism in which an endogenous gene is “knocked-out” by recombination with a partially homologous exogenous sequence which introduces a mutation within the endogenous sequence. Typically, the vector is directed at an endogenous target sequences which may be all or part of a gene of interest. The vector includes 5′ and 3′ flanking sequences which are homologous to the 5′ and 3′ ends of the target. Between the 5′ and 3′ flanking sequences is the sequence including the mutation. The mutation can be a termination mutation, frame-shift mutation, large deletion, or even the introduction of a new coding sequence which serves both to disrupt the endogenous gene and to act as a marker for successful homologous recombination. Knock-out vectors are further discussed below.

In other embodiments, the SID-1 coding sequences can be joined to regulatory regions and heterologous coding sequences to form a genetic construct or fusion vector which encodes a fusion protein. Fusion vectors and fusion proteins can be useful to increase the expression of the SID-1 protein, to increase the solubility of the SID-1 protein, and aid in the purification of the SID-1 protein (e.g., by acting as a ligand for affinity purification). A proteolytic cleavage site may be introduced at the junction of the SID-1 and non-SID-1 protein sequences so that the SID-1 protein can easily be separated from the fusion moiety. Typical fusion expression vectors include pGEX (Smith et al (1988), Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein, and vectors that fuse green fluorescent protein (GFP) or other fluorescent proteins to the target protein (Miller et al. (1999) Biotechniques 26:914, 921).

In another aspect, the present invention provides cell lines transformed with the nucleic acid molecules of the invention. As used herein, with respect to genetic engineering, the term “transform” means to introduce into a cell or an organism an exogenous nucleic acid or nucleic acid analog which replicates within that cell or organism, that encodes a polypeptide sequence which is expressed in that cell or organism, and/or that is integrated into the genome of that cell or organism so as to affect the expression of a genetic locus. The term “transform” is used to embrace all of the various methods of introducing such nucleic acids or nucleic acid analogs, including, but not limited to the methods referred to in the art as transformation, transfection, transduction, electroporation, ballistic injection, and the like.

Such cell lines can simply propagate these nucleic acids (e.g., when transformed with cloning vectors) or can express the polypeptides encoded by these nucleic acids (e.g., when transformed with expression vectors). Such transformed cell lines may be used to produce the SID-1 proteins and SID-1 fragments of the invention, or may be used in assays to screen for compounds that enhance, repress, agonize, or antagonize SID-1 expression or activity.

The transformed cells may be produced by introducing into a cell an exogenous nucleic acid or nucleic acid analog which replicates within that cell, that encodes a polypeptide sequence which is expressed in that cell, and/or that is integrated into the genome of that cell so as to affect the expression of a genetic locus. The transformation may be achieved by any of the standard methods referred to in the art as transformation, transfection, transduction, electroporation, ballistic injection, and the like. The method of transformation is chosen to be suitable to the type of cells being transformed and the nature of the genetic construct being introduced into the cells.

Preferred cell lines for transformation include bacterial cells (e.g., Escherichia coli), yeast cells (e.g., Saccharomyces cerevisiae), insect cells (e.g., Drosophila melanogaster Schneider cells), nematode cells (e.g., Caenorhabditis elegans), amphibian cells (e.g., Xenopus oocytes), rodent cells (e.g., Mus musculus (e.g., murine 3T3 fibroblasts), Rattus rattus, Chinese Hamster Ovary cells (e.g., CHO-K1)), and human cells (e.g., human skin fibroblasts, human embryonic kidney cells (e.g., HEK-293 cells), COS cells). Transformed mammalian cells useful in the invention include somatic cells, fetal cells, embryonic stem cells, zygotes, gametes, germ line cells and transgenic animal cells.

Appropriate cells may be transformed with any of the above-described genetic constructs in order to produce SID-1 proteins, including fragments of SID-1 proteins, fusion proteins of SID-1 proteins, or marker proteins under the control of a SID-1 regulatory region.

The cells may be transformed according to any method known in the art appropriate to the cell type being transformed. Appropriate methods can include those described generally in, e.g., Sambrook et al (1989), Molecular Cloning: A Laboratory Manual, Cold Spring Harbor Laboratories, New York; and Davis et al (1986), Basic Methods in Molecular Biology, Elsevier. Particular methods include calcium phosphate co-precipitation (Graham et al (1973), Virol. 52:456-467), direct micro-injection into cultured cells (Capecchi (1980), Cell 22:479-488), electroporation (Shigekawa et al (1988), BioTechniques 6:742-751), liposome-mediated gene transfer (Mannino et al. (1988), BioTechniques 6:682-690), lipid-mediated transduction (Felgner et al. (1987), Proc. Natl. Acad. Sci. USA 84:7413-7417), and nucleic acid delivery using high-velocity microprojectiles (Klein et al. (1987), Nature 327:70-73).

The present invention also provides for the production of transgenic non-human animal models in which wild type, allelic variant, chimeric, or antisense SID-1 sequences are expressed, or in which SID-1 sequences have been inactivated or deleted (e.g., “knock-out” constructs) or replaced with reporter or marker genes (e.g., “knock-in reporter” constructs). As used herein, the term “reporter gene” means any genetic sequence which, when expressed, has a biochemical or phenotypic effect which is detectable. Reporter genes are also known in the art as “marker” genes. The SID-1 sequences may be conspecific to the transgenic animal (e.g., nematode sequences in a transgenic nematode) or transpacific to the transgenic animal (e.g. nematode sequence in a transgenic fruit fly or mouse). In such a transgenic animal, the transgenic sequences may be expressed inducibly, constitutively or ectopically. Expression may be tissue-specific or organism-wide. Engineered expression of SID-1 sequences in tissues and cells not normally containing SID-1 gene product may confer systemic RNAi activity in an animal lacking endogenous RNAi activity. Ectopic or altered levels of expression of SID-1 sequences may alter cell, tissue and/or developmental phenotypes. Transgenic animals are useful as models of disorders arising from defects in SID-1 activity.

Transgenic animals are also useful for screening compounds for their effects on SID-1 activity. Transgenic animals transformed with reporter constructs may be used to measure the transcriptional effects of small molecules or drugs or physical perturbations on the expression of SID-1 genes and proteins in vivo. The transgenic animals of the invention, may be used to screen such compounds for therapeutic utility.

Animal species suitable for use in the animal models of the present invention include, but are not limited to, nematodes, insects such as Drosophila melanogaster, rats, mice, hamsters, guinea pigs, rabbits, dogs, cats, goats, sheep, pigs, and non-human primates (e.g., Rhesus monkeys, chimpanzees). For initial studies, transgenic rodents (e.g., mice) are preferred due to their relative ease of maintenance and shorter life spans. Transgenic non-human primates may be preferred for longer term studies due to their greater similarity to humans.

Using the nucleic acids disclosed and otherwise enabled herein, there are several available approaches for the creation of a transgenic animal. Thus, the enabled animal models include: (1) animals in which sequences encoding at least a functional fragment of a SID-1 gene has been recombinantly introduced into the genome of the animal as an additional gene, under the regulation of either an exogenous or an endogenous promoter element, and as either a minigene (i.e., a genetic construct of the SID-1 gene based on cDNA with introns removed) or a large genomic fragment; (2) animals in which sequences encoding at least a functional fragment of a SID-1 gene have been recombinantly substituted for one or both copies of the animal's endogenous SID-1 gene by homologous recombination or gene targeting; (3) animals in which sequences encoding a reporter gene have replaced the endogenous SID-1 gene by homologous recombination; (4) and “knock-out” animals in which one or both copies of the animal's SID-1 sequences have been partially or completely inactivated by the insertion, deletion or substitution of one or more nucleotides by homologous recombination. These and other transgenic animals of the invention are useful as models for understanding the mechanism of systemic RNA interference. These animals are also useful for screening compounds for their effects on the SID-1 gene and/or protein and for identifying other genes involved in RNA interference.

To produce an animal model (e.g., a transgenic nematode), a wild type or allelic variant SID-1 sequence or a wild type or allelic variant of a recombinant nucleic acid encoding at least a functional fragment of a SID-1 protein is preferably inserted into a germ line or stem cell using standard techniques of oocyte or embryonic stem cell microinjection, or other form of transformation of such cells. Alternatively, other cells from an adult organism may be employed. Animals produced by these or similar processes are referred to as transgenic. Similarly, if it is desired to inactivate or replace an endogenous SID-1 sequence, homologous recombination using oocytes, embryonic stem or other cells may be employed. Animals produced by these or similar processes are referred to as “knock-out” (inactivation) or “knock-in” (replacement) models.

For oocyte injection, one or more copies of the recombinant DNA constructs of the present invention may be inserted into the pronucleus of a just-fertilized oocyte. This oocyte is then reimplanted into a pseudo-pregnant foster mother. The live born animals are screened for integrants using standard DNA/mRNA analysis (e.g., from the tail veins of offspring mice) for the presence of the inserted recombinant transgene sequences. The transgene may be either a complete genomic sequence introduced into a host as part of a yeast artificial chromosome (YAC), bacterial artificial chromosome (BAC), or other chromosome DNA fragment; as a cDNA with either the endogenous promoter or a heterologous promoter; or as a minigene containing all of the coding regions and other elements found to be necessary for optimum expression.

To create a transgene, the target sequence of interest (e.g., a wild type or allelic variant of a SID-1 sequence) is typically ligated into a cloning site located downstream of a promoter element which will regulate the expression of RNA from the sequence. Downstream of the coding sequence, there is typically a polyadenylation sequence. An alternative approach to creating a transgene is to use an exogenous promoter and regulatory sequences to drive expression of the transgene. Finally, it is possible to create transgenes using large genomic DNA fragments such as YACs which contain the entire desired gene as well as its appropriate regulatory sequences.

Animal models may be created by targeting endogenous SID-1 sequences for homologous recombination. These targeting events can have the effect of removing endogenous sequence (knock-out) or altering the endogenous sequence to create an amino acid change associated with human disease or an otherwise abnormal sequence (e.g., a sequence which is more like the human sequence than the original animal sequence) (knock-in animal models). A large number of vectors are available to accomplish this and appropriate sources of genomic DNA for mouse and other animal genomes to be targeted are commercially available (e.g., GenomeSystems Inc., St. Louis, Mo.).

In another aspect, the present invention provides substantially pure preparations of SID-1 proteins. The proteins can be isolated from nematode cells or other cells endogenously expressing SID-1, using standard techniques such as immunoaffinity purification with the antibodies of the invention (see below), but are preferably isolated from the transformed cells of the invention, in which they may be expressed at higher levels and, optionally, as fusion proteins which are more easily isolated and/or purified.

In some embodiments, the SID-1 proteins comprise the entire translated sequence of the SID-1 coding region. Examples of such full-length SID-1 proteins include the C. elegans SID-1 protein disclosed as SEQ ID NO:2, as well as other SID-1 proteins, including alleles, variants, and functional equivalents thereof.

In other embodiments, the SID-1 proteins are SID-1 fragments. Such fragments include the structural domains of the SID-1 proteins, including the transmembrane and extracellular domains.

In certain embodiments, polypeptides are provided having at least 80%, and preferably at least 85%, 90% or 95% amino acid sequence identity with at least a structural domain of a SID-1 protein. Thus, in some embodiments, a polypeptide is provided having at least 80%, 85%, 90% or 95% amino acid sequence identity with a transmembrane domain of a SID-1 protein or an extracellular domain of a SID-1 protein. In some preferred embodiments, polypeptides are provided having at least 80%, 85%, 90% or 95% amino acid sequence identity with a SID-1 protein and having SID-1 activity. The ability of a protein to exhibit SID-1 activity can be measured by its ability to complement a SID-1 −/− mutant (e.g., a SID-1 knock-out mutant) and restore a normal or SID-1 +/+phenotype (e.g., to restore systemic RNA interference) in a cell otherwise capable of expressing SID-1 activity (e.g., a nematode cell from the SID-1 −/− mutant).

In certain embodiments, the polypeptides of the invention include a SID-1 sequence of at least 50 amino acid residues in length, and preferably at least 100, 200 or 300 amino acid residues in length. These polypeptides can include a SID-1 sequence which includes at least one transmembrane domain, at least one extracellular loop domain, or combinations thereof. In some preferred embodiments, the polypeptide has SID-1 activity.

In another aspect, the invention provides a substantially pure protein preparation including polypeptides having at least 80%, 85%, 90%, or 95% amino acid sequence identity with a SID-1 protein; at least a transmembrane domain of a SID-1 protein; or at least an extracellular domain of a SID-1 protein. In some embodiments, the substantially pure preparation includes a polypeptide having at least 80%, 85%, 90%, or 95% amino acid sequence identity with a SID-1 protein and having SID-1 activity in a cell capable of expressing SID-1 activity.

As used herein, the term “substantially pure” means a preparation which contains at least 60% (by dry weight) of the protein of interest, exclusive of the weight of other intentionally included compounds. Preferably the preparation is at least 75%, more preferably at least 90%, and most preferably at least 99%, by dry weight of the protein of interest, exclusive of the weight of other intentionally included compounds. Purity can be measured by any appropriate method, e.g., column chromatography, gel electrophoresis, amino acid compositional analysis or HPLC analysis. If a preparation intentionally includes two or more different proteins of the invention, a “substantially pure” preparation means a preparation in which the total dry weight of the protein of the invention is at least 60% of the total dry weight, exclusive of the weight of other intentionally included compounds. Preferably, for such preparations containing two or more proteins of the invention, the total weight of the proteins of the invention should be at least 75%, more preferably at least 90%, and most preferably at least 99%, of the total dry weight of the preparation, exclusive of the weight of other intentionally included compounds. Thus, if the proteins of the invention are mixed with one or more other compounds (e.g., diluents, detergents, excipients, salts, sugars, lipids) for purposes of administration, stability, storage, and the like, the weight of such other compounds is ignored in the calculation of the purity of the preparation.

In another aspect, the present invention provides substantially pure preparations of antibodies against SID-1 polypeptides, and methods of making such antibodies. As used herein, the term “antibody” is intended to embrace naturally produced antibodies, recombinantly produced antibodies, and antibody fragments such as Fab fragments, F(ab′)₂ fragments, Fv fragments, and single-chain Fv fragment (scFv).

The antibodies may be raised against the full-length SID-1 proteins, against fragments of the SID-1 proteins, or using any SID-1 peptide or epitope which is characteristic of the proteins and which substantially distinguishes them from other proteins. In at least some, the epitope is a protein sequence of at least 6-12, preferably 10-20, more preferably 15-30 consecutive amino acid residues of a SID-1 protein. Epitopes having a high predicted antigenicity can be identified by prediction of hydrophobicity, surface probability and antigenic index using standard programs, including GCG and MacVector (Genetics Computer Group, University of Wisconsin Biotechnology Center, Madison, Wis.; Accelrys Inc., San Diego, Calif.). See also, Jameson and Wolf (1988), Comput. Appl. Biosci. 4:181-186.

SID-1 immunogen preparations may be produced from crude extracts (e.g., microsomal fractions of cells expressing the proteins), from proteins or peptides substantially purified from cells which naturally or recombinantly express them or, for small immunogens, by chemical peptide synthesis. The SID-1 immunogens may also be in the form of a fusion protein in which the non SID-1 region is chosen for its adjuvant properties and/or the ability to facilitate purification.

The antibodies of the invention may be polyclonal or monoclonal, or may be antibody fragments, including Fab fragments, F(ab′)₂ fragments, Fv fragments, and single chain Fv fragments (scFv). In addition, after identifying useful antibodies by the method of the invention, recombinant antibodies may be generated, including any of the antibody fragments listed above, as well as chimeric and/or humanized antibodies based upon non-human antibodies to the SID-1 proteins. In light of the present disclosure of SID-1 proteins, as well as the characterization of other SID-1 proteins enabled herein, one of ordinary skill in the art may produce the above-described antibodies by any of a variety of standard means. For an overview of antibody techniques, see Antibody Engineering, 2nd Ed., Borrebaek, ed., Oxford University Press, Oxford (1995).

As a general matter, monoclonal anti-SID-1 antibodies can be produced by first injecting a mouse, rabbit, goat or other suitable animal with a SID-1 immunogen in a suitable carrier or diluent. Carrier proteins or adjuvants can be utilized, and booster injections (e.g., bi- or tri-weekly over 8-10 weeks) can be employed as necessary. After allowing for development of a humoral response, the animals are sacrificed and their spleens are removed and resuspended in an appropriate buffer (e.g., phosphate buffered saline). The spleen cells serve as a source of lymphocytes, some of which will produce antibodies of the appropriate specificity. These cells are then fused with an immortalized cell line (e.g., a myeloma), and the products of the fusion are plated into tissue culture wells in the presence of a selective agent (e.g., HAT). The wells are serially screened and replated, selecting cells making a useful antibody each time. Typically, several screening and replating procedures are carried out until the wells contain single clones which are positive for antibody production. Monoclonal antibodies produced by such clones may be purified by standard methods such as affinity chromatography using Protein A Sepharose, by ion-exchange chromatography, or by variations and combinations of these techniques.

Antibodies of the invention may be used in a variety of applications. For example, antibodies may be used in a purification process (i.e., immunoaffinity purification) for SID-1 proteins, in assays to detect the presence or level of SID-1 protein in cells or animals (e.g., in a diagnostic test for a SID-1-related disorder), or in assays to measure the presence or level of SID-1 expression in transformed cells (e.g., in assays for regulators of SID-1 expression, in Western blotting to identify cells expressing SID-1 proteins, or in immunocytochemistry or immunofluorescence techniques to establish the cellular or extracellular location of SID-1 proteins).

The antibodies of the invention may be bound or conjugated with other compounds or materials for diagnostic and/or therapeutic uses. For example, they may be coupled to labels such as radionuclides, fluorescent compounds (e.g., rhodamine), or enzymes for imaging or therapy. The labels maybe bound to the antibodies covalently or non-covalently.

In another aspect, the invention provides kits for detecting at least an epitope of a SID-1 protein. The kits include an anti-SID-1 antibody and a means for detecting the antibody. The means for detecting the antibody can be a detectable label bound to the antibody or secondary antibodies for detecting the anti-SID-1 antibodies (e.g., a labeled goat anti-rabbit-Ig antibody as a secondary antibody for detecting a rabbit anti-SID-1 antibody).

In another aspect, the invention provides a method for reducing the expression of a target gene in a cell comprising the steps of introducing a nucleic acid vector comprising a sid-1 sequence into the cell and introducing a double-stranded RNA molecule having a sequence complementary to the target gene, wherein the SID-1 sequence encodes a polypeptide having SID-1 activity. As used herein, the term “expression” refers to the process by which a coding sequence of a gene is transcribed into a primary mRNA transcript, the primary mRNA transcript is processed into a mature mRNA, and the mature mRNA is translated into a protein. Expression may optionally include post-translation modifications of the resulting polypeptide. As used herein, the terms “increase” and “decrease” mean, respectively, statistically significantly increase (i.e., p<0.1) and statistically significantly decrease (i.e., p<0.1).

A decrease in the expression of a target gene in a cell can be determined using a variety of methods known to those of skill in the art. For example, immunostaining with monoclonal antibodies directed against the protein of interest can be used to examine the expression of a protein of interest in a whole cell. Total cellular proteins can be extracted from the cell, and subjected to electrophoresis and Western blotting. These and other methods are described in, for example, Current Protocols in Molecular Biology, Vol. I, John Wiley & Sons, Inc., New York, and Sambrook et al. (1989) Molecular Cloning: A Laboratory Manual, Cold Spring Harbor Laboratories, New York).

The vector encoding the sid-1 sequence can be introduced into cells using methods for transformation of cells as described above herein.

The interfering double-stranded RNA construct can be introduced into the cell using methods described above suitable for transforming cells. Alternatively, the RNA construct can be prepared using in vitro methods such as cell-free transcription or can be isolated from cells expressing the double-stranded RNA molecule. The double-stranded RNA molecule can be administered directly to the cells in solution in the culture medium.

Small double-stranded RNAs from 20-30 nucleotides in length that act as small interfering RNAs (siRNAs) can be administered directly to the cells in solution or may be transfected into cells as described above. Constructs that express small RNAs that can fold into a double-stranded form suitable for cleavage by dicer into siRNAs can be expressed from expression vectors or viral vectors (see, e.g., Sui et al (2002) Proc. Natl. Acad. Sci. USA 99:5515-20). These can be transfected into cells in culture or into animals. Small RNAs can also be injected into animals, administered in an aerosol, oradministered directly to the target tissue by injection or topological application.

In another aspect, the invention provides a method for reducing the expression of a target gene in a population of cells, comprising the steps of introducing a nucleic acid vector comprising a sid-1 sequence into at least a portion of the population of cells and introducing a double-stranded RNA molecule having a sequence complementary to the target gene, wherein the sid-1 sequence encodes a polypeptide having SID-1 activity.

In another aspect, the invention provides a method for reducing the expression of a target gene in an animal, the method comprising introducing a nucleic acid vector comprising a sid-1 sequence into the animal, and introducing a double-stranded RNA molecule having a sequence complementary to the target gene, wherein the sid-1 sequence encodes a polypeptide having SID-1 activity.

Systemic RNAi was observed in C. elegans using a transgenic strain, HC57, which expresses a reporter gene encoding green fluorescent protein (GFP) in both the pharynx and the body wall muscle. The HC57 strain contains a transgene that expresses GFP under control of the pharynx-specific myo-2 promoter. HC57 also expresses two other “target” GFP transgenes: myo-2::GFP, which is expressed in pharyngeal muscle cells, and which should be susceptible to RNAi initiated in the pharyngeal cells, and myo-3::GFP-NLS, which is expressed in the body wall muscles (localized to the nuclei), and which should be susceptible to RNAi spreading from the pharynx to the body wall muscle. Thus, the HC57 strain contains a reporter that will be silenced in the pharynx when RNAi is initiated in the pharynx, and a reporter in the body wall muscle that is silenced, when RNAi initiated in the pharynx spreads to the adjacent body wall muscle.

The HC57 strain also expresses double-stranded RNA (dsRNA) hairpin molecules in the pharynx, which act as the interfering RNA. Cell-autonomous RNAi was observed in the pharynx and found to be highly but incompletely penetrant and temperature sensitive. Spreading of the RNAi effect was observed by examining gene silencing in the body wall muscle, which does not express an interfering dsRNA construct, but where the cells are in contact with the cells of the pharynx. Systemic RNAi observed in the body wall muscle was found to be position-dependent and temperature dependent. The silencing of GFP expression in both the pharynx and the body wall muscle was found to be dependent on rde-1, verifying that an RNAi mechanism was responsible for the observed reduction in gene expression.

The HC57 strain was exploited to screen for mutations that permitted cell-autonomous gene silencing in pharynx, but inhibited the spreading of the RNAi effect to the adjacent body wall muscle. Detection of RNAi was augmented by incorporating bacteria-mediated RNAi in pharynx into the experiment. HC57 animals were grown on E. coli expressing a GFP double-stranded RNA hairpin construct with a loop region homologous to unc-22. Animals grown on these bacteria display partial silencing of GFP in the pharynx, complete silencing of GFP in the body wall muscle, and a strong Unc-22 twitching phenotype caused by silencing of the unc-22 gene in body wall muscle.

To identify mutants specifically defective in systemic RNAi, animals were mutagenized and mated, and an F₂ screen was conducted for mutants resistant to RNAi in body wall muscle. These mutants did not display silencing of GFP in body wall muscle, and did not display the strong Unc-22 twitching phenotype. Mutants obtained in the screen were designated Systemic RNA Interference Defective (sid), and defined three major complementation groups (sid-1, sid-2, and sid-3). The sid-1 genes are discussed herein.

To investigate the sid-1 phenotype in greater detail, dsRNAs targeting different classes of mRNAs were introduced into a reference allele of sid-1 mutant animals (qt2) by a variety of methods. To verify that the phenotype of sid-1 animals was not due to a defect in RNAi limited to body wall muscles, a transgene expressing GFP ds RNA was introduced into the germine of sid-1 mutant animals. This transgene could direct cell-autonomous RNAi in body wall cells, confirming that the sid-1 phenotype could be attributed to a failure of systemic RNAi.

Systemic RNAi was also assayed by injecting ds RNA into the intestines of sid-1 animals. Injection of mex-3 dsRNA into the intestine of adult wild-type hermaphrodites targets mex-3 transcripts in germ cells, producing a maternal effect mex-3 lethal phenotype in F₁ progeny. Similar injections into sid-1 hermaphrodites produced only viable F₁ progeny, demonstrating that in sid-1 mutants the RNAi response cannot spread from the intestine to the germ cells. When mex-3 was injected directly into the gonad of sid-1 animals, the lethal mex-3 phenotype was observed, indicating that cell-autonomous RNAi was not affected in sid-1 mutants. It was also noted that RNAi did not appear to spread throughout the gonad of sid-1 mutants, as 100% lethality was only observed in sid-1 mutants when the mex-3 construct was injected into both arms of the gonad.

Further experiments examined the transmission of silencing by systemic RNAi from parent to progeny. Injecting the intestine or gonad of wild-type hermaphrodites with unc-22 dsRNA efficiently produces an Unc-22 twitching phenotype among the progeny. Similar injections into sid-1 hermaphrodites demonstrated that sid-1 is required for RNAi-mediated silencing in the progeny of injected animals. Because the phenotype is being assayed in the progeny of injected animals, it was possible to examine whether supplying sid-1 to the progeny was capable of restoring the systemic RNAi effect. sid-1 hermaphrodites were injected with unc-22 dsRNA and subsequently crossed with wild-type males to determine whether the resulting heterozygous progeny were susceptible to RNAi. The sid-1 embryos were susceptible, but the penetrance of the RNAi effect depended on the site of injection. Injecting the hermaphrodites in the gonad was considerably more efficient than injecting the intestine. The strong response from gonad injections suggests that embryos that inherit ds RNA or an autonomous RNAi response require sid-1 function to transmit the effect to their somatic tissues. The weak response from intestine injected hermaphrodites cross to wild-type males suggests that sid-1 function is required for efficient transmission of the RNAi response from the intestine to the germline.

In another experiment directed to examine the transmission of RNAi between parent and progeny, the ability of a heterozygous sid-1/+ hermaphrodite to transmit silencing to sid-1/sid-1 progeny was examined. Heterozygous hermaphrodites were injected in either the intestine or both gonad arms. Nearly all homozygous sid-1 F1 progeny displayed the Unc-22 phenotype, indicating that the RNAi response initiated in the injected parent can spread to the mutant F1 progeny. This result suggests that maternal sid-1 function is sufficient to initially spread RNAi throughout the mutant embryo. The results of these experiments are summarized in Table 1.

TABLE 1
Characterization of sid-1 systemic RNAi resistance. Progeny
of sid-1(qt2) and wild-type (WT) worms exposed to mex-3 and
unc-22 dsRNA by various methods were scored for RNAi phenotypes.
Asterisks indicate that only cross progeny were scored.

| dsRNA delivery (hours after | | | | |
|---|---|---|---|---|
| injection) | Percent embryonic lethal | | | |
| (A) mex-3 RN Ai | Wild-type N2 | | sid-1 (qt2) | |
| Bacteria-mediated (NA) | 100 | (615) | 1 | (535) |
| Intestine (12.5 to 24.5) | 86 | (665) | 2 | (782) |
| | Percent twitching progeny | | | |
| (B) unc-22 RNAi | Wild-type N2 | | sid-1 (qt2) | |
| Bacteria-mediated (NA) | 100 | (394) | 0 | (363) |
| Intestine (11 to 23) | 68 | (701) | 0 | (563) |
| Intestine crossed to WT males | 70 | (497)* | 14 | (571)* |
| (7.5 to 31.5) | | | | |
| Anterior gonad arm (15.5 to 42.5) | 89 | (688) | 0 | (981) |
| both gonad arms (7 to 40.5) | 80 | (886) | 2 | (1050) |
| Both gonad arms crossed to WT | 99 | (206)* | 63 | (380)* |
| males (12 to 24) | | | | |

| | |
|---|---|
| | sid-1(qt2) dpy-11/++I |
| | (Dpy-11 progeny scored) |
| Intestine (9.5 to 24.5) | 96 (147) |
| Both gonad arms (9.5 to 24.5) | 98 (127) |

Because sid-1 is a transmembrane protein and is required for systemic RNAi, it is predicted to be required for the import or export of a systemic RNAi signal. To determine whether SID-1 is required cell autonomously to import a bacteria-mediated RNAi signal or whether it can function nonautonomously to deliver a signal from a neighboring cell, sid-1 genetic mosaics were analyzed. A sid-1 expressing a GFP target gene under control of the myo-3 promoter was injected with sid-1 gDNA and a second construct expressing the red fluorescent protein, DsRED2, under the control of the myo-2 promoter to produce extrachromosomal DNAs that rescue sid-1 and express dsRed2 in body-wall muscle cells. Because extrachromosomal arrays are mitotically unstable, mosaic animals were produced composed of cells that expressed DsRED2 and sid-1 and those that did express either protein. Thus, DsRED2 served as a marker for muscle cells which have either retained or lost expression of sid-1.

The mosaic worms were exposed to bacteria expressing GFP dsRNA. Because the GFP construct was under control of the myo-3 promoter, it was expressed only in body wall, and cells where silencing was observed had received the silencing information through systemic RNAi resulting from contact with the ingested dsRNA. DsRED2 expression was examined at the boundaries between cells that showed silencing of GFP and those that did not. Cells that were sensitive to systemic RNAi as evidenced by silencing of GFP had retained expression of DsRED, and therefore also retained SID-1, while those that were resistant to silencing of RNAi as evidenced by expression of GFP had lost expression of DsRED, and had therefore lost expression of SID-1. Thus, expression of SID-1 is required for the uptake or processing of a systemic RNAi signal.

The location and timing of expression of SID-1 were also examined. GFP transgenes under control of the sid-1 promoter were introduced into wild-type animals. Transgenes containing only the sid-1 promoter region fused to GFP were expressed in late embryos and were detected in nearly all non-neuronal cell types through adulthood. The highest levels of GFP expression in the adult were observed in cells and tissues in direct contact with the environment, such as the digestive system (pharynx, intestine, rectum), the excretory cell, the proximal gonad and spermatheca, and the phasmids. Another reporter construct was used in which GFP was fused to the C-terminus of the SID-1 protein, rather than replacing it entirely. This construct was capable of restoring function in sid-1 mutant animals, indicating that the expression pattern detected with the construct was representative of the endogenous expression pattern of sid-1. The fusion protein was detected in the cytoplasm, with significant enrichment of localization at the cell periphery.

Examination of the function of sid-1 in cultured nematode cells confirms the role of sid-1 in the import and/or processing of a systemic RNAi signal. Disassociated embryonic cells in culture were exposed to GFP double-stranded RNA (1 μg/1 ml, 2 μg/ml, 5 μg/ml 10 μg/ml) in the culture medium. GFP expressed from transgene under control of the myo-3 promoter was strongly inhibited in wild-type cells while sid-1 mutant cells maintained strong GFP expression.

The molecular identity of sid-1 was ascertained by conventional methods for mapping and cloning gene sequences (see, e.g., C. elegans, a Practical Approach, I.A. Hope, Ed., Oxford Univ. Press, New York, 1999). The molecular sequences of sid-1 genes obtained from several sid-1 mutants were compared with the molecular sequence of the wild-type gene to verify the identity of the sid-1 gene as the source of the sid-1 phenotype. Sequencing of the cloned gene revealed a reading frame encoding a predicted 776 amino acid transmembrane protein. The predicted protein has a signal peptide and eleven potential transmembrane domains. The presence of conserved transmembrane domains and the requirement of sid-1 for transmitting the RNAi signal are consistent with sid-1 acting as a pore required for active or passive transport of an RNAi signal.

Expression of SID-1 in cultured Drosophila S2 cells provided further indication that SID-1 is involved in the transport of the systemic RNAi signal into cells. S2 cells were transfected with a plasmid constitutively expressing SID-1 or with a control plasmid that did not express SID-1. The cells were exposed to an interfering RNA construct at a variety of concentrations corresponding to a linear range. The mass of dsRNA recovered from cells expressing SID-1 was greater in all cases than the mass of dsRNA recovered from cells transfected with the control plasmid. The cells were exposed to a specific dsRNA at a variety of concentrations corresponding to a linear range. The treated cells were washed extensively and the mass of associated dsRNA was determined by reverse transcription and quantitative polymerase chain reaction using the Drosophila gapdh (glyceraldehyde phosphate dehydrogenase) gene as an internal reference standard. Whereas it is known that at high concentrations, dsRNA will be taken up non-specifically by Drosophila 82 cells, it was observed that sid-1 expression resulted in a greater mass of dsRNA recovery at the lower range.

### EXAMPLES

### 1. Strains and Alleles

Strains and alleles used were BC1230 (dpy-18(e364)/eT1 III; sDf27 unc-46(e17)/eT1 V), CB4856 (Hawaiian polymorphic strain), HC46 (ccIs4251[myo-3::GFP-NLS (nuclear localized, myo-3:: GFP-MITO (mitochondrial localization)] I; mIs11[myo-2::GFP] IV), HC57 (qtIs3 [myo-2::GFP dsRNA] III in HC46 background), MT2583 (dpy-11(e224) nDf32 V/eT1(III; V)), dpy-11(e224) V, him-5(e1490) V, him-8 (e1489) IV, rde-1(ne219) V, sid-1(qt2) V. Many strains were obtained from the C. elegans genetic stock center (Caenorhabditis Genetics Center, University of Minnesota, 6-160 Jackson Hall, 321 Church Street S.E., Minneapolis, Minn. 55455).

### 2. Plasmid Construction

The myo-2::GFP dsRNA plasmid pHC168 was produced by inserting the GFP hairpin (with an unc-22 fragment loop) Age I (blunted)/NotI fragment from pPD126.25 into the pPD118.33 BspEI (blunted)/NotI vector. The myo-3::GFP dsRNA plasmid pHC172 was produced by inserting the GFP hairpin KpnI/NotI fragment from pPD126.25 into the pPD115.57 KpnI/EcoRI vector using a Nod to EcoRI linker. The myo-3::DsRED2 plasmid pHC183 was constructed by inserting the KpnI/NotI DsRed2 fragment from pDsRED2-N1 (BD Biosciences Clontech) into the pPD115.57 KpnI/EcoRI vector using a NotI to EcoRI linker. The pPD vectors (Miller et al. (1999) Biotechniques 26:914, 920) were gifts from A. Fire.

### 3. GFP Reporter Construction

sid-1::GFP reporters were made by ligation of PCR fragments. sid-1 promoter and full genomic coding region up to the last amino acid were amplified from N2 genomic DNA with the primers (a) 5′-ggtcatgagagggtcgagag-3′ and (b) 5′-aacgCCTAGGgaaaatgttaatcgaagttttgcgtgt-3′ (uppercase=AvrII site). GFP with an unc-54 3′ untranslated region (UTR) was amplified from the plasmid pPD95.75 using primers (c) 5′-ctaaGCTAGCatgagtaaaggagaagaacttttcact-3′ (uppercase=NheI site) and (d) 5′-tcaccgtcatcaccgaaac-3′. The two amplified fragments were gel purified using the Qiaquick kit (Qiagen) and then ligated together in the presence of AvrII, NheI and T4 DNA Ligase. The desired sid-1::C-GFP ligation fragment was gel purified using the Zymoclean kit (Zymo Research) and injected as described below. The sid-1 pro::GFP DNA fragment was produced by first amplifying the sid-1 promoter with the primers (e) 5′-aaaaactgcagggtcatgagagggtcgagag-37 (underline=same as primer (a)) and (f) 5′-acgcGGATCCggaaaaatgaggagttttaatttc-3′ (uppercase=BamHI site). Secondly, GFP with anSV40 NLS and unc-54 3′UTR was amplified from the plasmid pPD95.67 using primer (g) 5′-tggatacgctaacaacttggaa-3′ and primer (d). Finally, both fragments were digested with BamHI (the second fragment has a BamHI site in a multiple cloning site), gel purified (Qiaquick kit) and ligated together with T4 DNA Ligase. The desired 2.75 kb fragment was then gel purified (Zymoclean kit) and injected as described below.

### 4. sid-1cDNA Isolation

First strand cDNA was prepared from young adult poly-A+ worm RNA, and partial cDNA clones were isolated by PCR based on the C04F5.1 gene prediction. A clone containing the 3′ end of the cDNA was isolated by using an upstream primer (5′-gcggaaatcgttgattcttc-3′) and oligo-dT22 (with a T7 RNA polymerase binding site). A clone containing the 5′ end of the cDNA was isolated using a SL1 splice leader sequence (5′-gtttaattacccaagtttgag-3′) and a downstream primer (5′-gtgccataaatcgtgggaac-3′), The SL1 reaction produced a single product, while the other reaction produced one major band and many very minor ones, presumably due to non-specific priming of the oligo-dT22. These PCR fragments were cloned into pCR4 Blunt TOPO (Invitrogen) and sequenced using ABI Prism BigDye Terminator Cycle Sequencing Ready Reaction Kit and an ABI Prism 3100 DNA Analyzer (Applied Biosystems).

### 5. Identification of sid-1 Mutations

PCR products from genomic DNA samples of sid-1 mutants were amplified using the oligonucleotide primers 5′-ggtcatgagagggtcgagag-3′ and 5′-gcaaacgagcaattgtgaag-3′. Various primers were used for DNA sequencing (as above). All mutations sequenced were confirmed by sequencing two independent PCR products.

### 6. Transformation of RNAi Transgenes

pHC168 (100 μg/ml) and a 71-mer oligonucleotide (1 mg/ml) were injected into the germline of the HC46 strain. F1 transformants were identified by myo-2::GFP RNAi, and an integrated line was recovered (qtIs3). pHC172 (50 μg/ml) and the dominant transformation marker pRF4 (rol-6(su1006)) (50 μg/ml) were co-injected into the germline of ccIs4251; sid-1(qt2) adult hermaphrodites. To score for autonomous myo-3::GFP RNAi caused by pHC172, injected worms and their progeny were grown at 20° C.

### 7. Genetic Mosaic Analysis

sid-1 rescue fragment (15 μg/ml) (PCR-amplified from N2 genomic DNA with primers listed above in Identification of sid-1 mutations), pHC183 (25 μg/ml), and pRF4 (25 μg/ml) were co-injected into ccIs4251; sid-1 (qt2) hermaphrodite germlines, and F2 rescued lines were isolated. Four independent lines of transformed hermaphrodites were allowed to lay eggs on a small amount of OP50 E. coli for one day at 20° C. and were then removed. A large volume (˜100 μl) of bacteria expressing GFP dsRNA was then added to the F1 progeny, and sid-1 mosaic, Rol hermaphrodites were scored as adults for cell autonomy of sid-1. GFP reporters sid-1 pro::GFP fragment (25 μg/ml) and pRF4 (25 μg/ml) were co-injected into N2 hermaphrodite germlines. sid-1::C-GFP fragment (15 μg/ml) and pRF4 (25 μg/ml) were co-injected into N2 worms and into ccIs4251; sid-1(qt2) worms.

### 8. Bacteria-Mediated RNAi

For all bacteria-mediated RNAi experiments hairpin plasmids were used with HT115(DE3) E. coli that have an inducible T7 RNA polymerase L. Timmons, et al. (2001) Gene 263, 103. Frozen glycerol stocks of plasmid strains were used to inoculate 1 liter of Terrific Broth, 50 μg/ml carbenicillin, and 12.5 μg/ml tetracycline. Cultures were grown approximately 24 hours at 37° C. and pelleted. Pellets were resuspended in four milliliters of 0.5×M9 (3) containing 15% glycerol per one gram of pellet and stored at −80° C. Thawed cultures were spotted on NG (T. Stiemagle, in C. elegans, a Practical Approach, I.A. Hope, Ed. (Oxford Univ. Press, New York, 1999), chap. 4. plates, and embryos were placed on the plates to score their progeny for mex-3 (25° C.) and mex-6 (15° C.) RNAi. For GFP (20° C.), unc-22 (25° C.), and unc-54 (25° C.), L4 larvae or young adults were placed on thawed food and their progeny were scored as IA/young adults for RNAi. Plasmids used were pPD126.25 (GFP, unc-22 loop), pPD128.117 (unc-22, GFP loop), pPD128.86 (unc-54, unc-54 loop), pHC169 (mex-6, Kan R loop), and pHC171 (mex-3, GST loop). These plasmids have a T7 promoter on one side of the hairpin and lac and T3 promoters on the other side. The loop sequence and the intended hairpin sequence were both able to induce an RNAi response when using pPD 126.25 and pPD 128.117. This suggests that dsRNA corresponding to the loop is produced, likely by unintended transcription from the lac promoter on the other side of the hairpin, creating an RNA molecule that can anneal to the intended transcript. This is more likely than a transitive RNAi phenomenon T. Sijen et al. (2001) Cell 107: 465 because bacteria-mediated RNAi with pPD126.25 causes the Unc-22 phenotype in wild-type N2 worms. Additionally, expression from the lac promoter as well as the T7 promoter is likely, since there is presumably leaky expression of the inducible T7 RNA polymerase, which also uses the lac promoter.

### 9. dsRNA Preparation and Microinjection

Double-stranded mex-3 RNA was made by in vitro transcription with T7 RNA polymerase and linearized pHC170 (mex-3 hairpin in pCR4Blunt TOPO (Invitrogen)). Double-stranded unc-22 hairpin RNA was made by in vitro transcription with T7 RNA polymerase and linearized pPD128.117. dsRNA was annealed by heating to 90° C. for two minutes and cooling one degree every eight seconds until reaching 25° C. All dsRNA injections used 1 mg/ml dsRNA in water and were assayed at 25° C. unless stated otherwise in the text. sid-1 (qt2) worms were injected before N2 wild-type.

### 10. EMS Mutagenesis

Synchronized parental HC57 young adult hermaphrodites were mutagenized with 25 mM ethyl methanesulfonate for four hours. Fifty-four pools of ˜6,000 synchronized F1 worms were obtained by hypochlorite treatment, and a synchronous F2 population was obtained from the F1s. The synchronized F2 L1 larvae were placed on pPD 126.25 HT115(DE3) E. coli at 20° C., and adult F2 worms that were resistant to myo-3::GFP RNAi, but still sensitive to cell-autonomous myo-2::GFP RNAi were recovered. Additionally, many F2 animals showed bright GFP expression in body-wall muscles and pharyngeal muscles, consistent with a general defect in RNAi. These were not selected.

### 11. Systemic RNAi Assay

Visualization of systemic RNAi of GFP is enhanced by starvation. This is likely due to the perdurance of GFP in normally growing worms. Five L4s were placed on an OP50 seeded 60 mm NG plate at 20° C. Two days after the plate starved, the starved plate was chunked to a fresh plate at 20° C. The starved larvae that became adults were observed.

### 12. Microscopy

Images were acquired using Openlab 3 software (Improvision) and Zeiss Axiophot and Axioskop microscopes. Images for deconvolution analysis were acquired with an Olympus IX70 microscope using 0.3 μM steps in the Z-axis and then deconvolved using softWoRx 2.50 software (Applied Precision). For display, three consecutive deconvolved images are overlaid. Confirmed polymorphisms used to map sid-1. Primers, restriction enzymes, and expected restriction fragment sizes for snip-SNPs used are listed. One polymorphism (for cosmid C03A7) was a PCR polymorphism (six bands (N2) vs. five bands (HA), likely due to a different number of DNA repeats). Some polymorphisms were found at http://www.genome.wustl.edu/gsc/Projects/C.elegans and the others were provided by Wicks, et al (2001) Nat. Genet. 28, 160.

### 13. Function of sid-1 in Cultured Nematode Cells

Dissociated C. elegans embryonal cells were cultured according to the methods described in Christensen et al. (Neuron (2002) 33, 503-514). Interfering RNA obtained by in vitro transcription of an inverted repeat encoding GFP and annealing of the single stranded regions. The GFP dsRNA was added to the culture medium at a concentration of 5 μg/mL. Cells were observed 24-48 hours after addition of the interfering RNA and scored for expression of GFP.

### 14. Uptake of dsRNA in Drosophila S2 Cells

Drosophila S2 cells (obtained from Invitrogen) were transfected by calcium phosphate treatment with pHC235, which expresses C. elegans SID-1 under the control of the actin 5 promoter, or with a control plasmid. The transfected cells were challenged with an interfering dsRNA construct at a linear set of concentrations. The relative level of dsRNA recovered after extensive washing was determined by quantitative RT-PCR. At all concentrations between 0.05 and 500 pg/ml the expression of SID-1 increased the mass of dsRNA recovered from the cells by 10-100 fold. Pretreating naïve cells with 1 μg of non-specific DNA or dsRNA reduces apparent background by another ten-fold.

What is claimed is:

1. An isolated nucleic acid comprising SEQ ID NO: 1 or a sequence complementary to SEQ ID NO: 1.

2. An isolated nucleic acid comprising a nucleotide sequence selected from the group consisting of:
(a) a sequence encoding a systemic RNA interference deficient-1 protein (SID-1 protein);
(b) a sequence encoding at least a transmembrane domain of a SID-1 protein, wherein said transmembrane domain is comprised of a polypeptide of SEQ ID NO: 2 selected from the group consisting of residues 425-451, 481-502, 509-541, 546-571, 575-599, 601-621, 633-655, 659-681, 692-712 and 742-766;
(c) a sequence encoding at least an extracellular domain of a SID-1 protein, wherein said extracellular domain comprises residues 19-314 of SEQ ID NO: 2; and
(d) a sequence complementary to any one of the sequences of (a)-(c).

3. An isolated nucleic acid encoding a polypeptide having at least 80% amino acid sequence identity with a polypeptide selected from the group consisting of:
(a) SEQ ID NO: 2;
(b) at least a transmembrane domain of a SID-1 protein, wherein said transmembrane domain is comprised of a polypeptide of SEQ ID NO: 2 selected from the group consisting of residues 425-451, 481-502, 509-541, 546-571, 575-599, 601-621, 633-655, 659-681, 692-712 and 742-766; and
(c) at least an extracellular domain of a SID-1 protein, wherein said extracellular domain comprises residues 19-314 of SEQ ID NO: 2.

4. An isolated nucleic acid encoding a polypeptide having at least 80% sequence identity with a SID-1 protein, wherein said SID-1 protein has SID-1 activity.

5. An isolated nucleic acid comprising:
(i) a nucleotide sequence encoding a polypeptide having at least 80% amino acid sequence identity with SEQ ID NO:2; and
(ii) a heterologous regulatory region operably joined to said sequence such that said sequence is expressed.

6. A kit for detecting at least a portion of a SID-1 nucleic acid comprising an isolated nucleic acid according to claim 1 and a means for detecting said isolated nucleic acid.

7. The kit according to claim 6, wherein said means for detecting said isolated nucleic acid comprises a detectable label bound thereto.

8. A kit according to claim 6, wherein said means for detecting said isolated nucleic acid comprises a labeled secondary nucleic acid which hybridizes to said isolated nucleic acid.

9. A vector comprising an isolated nucleic acid according to claim 1.

10. A vector comprising a genetic construct capable of expressing a nucleic acid according to claim 1.

11. The vector according to claim 10, wherein said nucleic acid is operably joined to an exogenous regulatory region.

12. The vector according to claim 10, wherein said nucleic acid is operably joined to heterologous coding sequences to form a fusion vector.

13. A vector comprising an isolated nucleic acid according to claim 1 operably joined to a reporter gene.

14. A cell transformed with a nucleic acid according to claim 1.

15. A cell transformed with a genetic construct capable of expressing a nucleic acid according to claim 1.

16. The cell according to claim 15, wherein said nucleic acid is operably joined to heterologous sequences to encode a fusion protein.

17. The cell according to claim 15, wherein said cell is selected from the group consisting of bacterial cells, yeast cells, insect cells, nematode cells, amphibian cells, rodent cells, and human cells.

18. The cell according to in claim 15 wherein said cell is selected from the group consisting of mammalian somatic cells, fetal cells, embryonic stem cells, zygotes, gametes, germ line cells, and transgenic animal cells.

19. A non-human transgenic animal, wherein a genetic construct has introduced a modification into a genome of said animal, or an ancestor thereof, and wherein said modification is selected from the group consisting of insertion of a nucleic acid encoding at least a fragment of a SID-1 protein, inactivation of an endogenous SID-1 protein, and insertion by homologous recombination of a reporter gene operably linked to SID-1 regulatory elements.

20. The animal according to claim 19, wherein said modification is insertion of a nucleic acid encoding a polypeptide selected from the group consisting of a SID-1 protein, at least a transmembrane domain of a SID-1 protein, and at least an extracellular domain of a SID-1 protein.

21. The animal according to claim 20, wherein said animal is selected from the group consisting of rats, mice, hamsters, guinea pigs, rabbit, dogs, cats, goats, sheep, pigs, and non-human primates.

22. A substantially pure protein preparation comprising a polypeptide selected from the group consisting of:
(a) a SID-1 protein;
(b) at least a transmembrane domain of a SID-1 protein; and
(c) at least an extracellular domain of a SID-1 protein.

23. The substantially pure protein preparation according to claim 22, wherein said polypeptide is selected from the group consisting of:
(a) SEQ ID NO:2;
(b) a sequence comprising a polypeptide encoding residues 314-339, 425-451, 481-502, 509-541, 546-571, 575-599, 601-621, 633-655, 659-681, 692-712, or 742-766 of SEQ ID NO:2; and
(c) a sequence comprising a polypeptide encoding residues 19-314 of SEQ ID NO:2.

24. A substantially pure protein preparation comprising a polypeptide having at least 80% amino acid sequence identity with a polypeptide selected from the group consisting of:
(a) a SID-1 protein;
(b) at least a transmembrane domain of a SID-1 protein; and
(c) at least an extracellular domain of a SID-1 protein.

25. A substantially pure protein preparation comprising a polypeptide having at least 80% amino acid sequence identity with a SID-1 protein and having SID-1 activity in a cell capable of expressing SID-1 activity.

26. A substantially pure antibody preparation comprising an antibody raised against a SID-1 polypeptide.

27. The substantially pure antibody preparation according to claim 26, wherein said antibody is a monoclonal antibody.

28. The substantially pure antibody preparation according to claim 26, wherein said antibody is an antibody fragment selected from the group consisting of an Fab fragment, an F(ab′)2 fragment, an Fv fragment, and a single-chain Fv fragment (scFv).

29. A kit for detecting at least an epitope of a SID-1 protein comprising an anti-SID-1 antibody of claim 26 and a means for detecting said antibody.

30. The kit according to claim 29, wherein said means for detecting said anti-SID-1 antibody comprises a labeled secondary antibody which specifically binds to said anti-SID-1 antibody.

31. The kit according to claim 29, wherein said means for detecting said anti-SID antibody comprises a detectable label bound thereto.

32. A method for reducing the expression of a target gene in a cell, the method comprising:
(a) introducing a nucleic acid vector comprising a SID-1 sequence; and
(b) introducing a double-stranded RNA molecule having a sequence complementary to the target gene,
wherein the SID-1 sequence encodes a polypeptide having SID-1 activity.

33. A method for reducing the expression of a target gene in a population of cells, the method comprising:
(a) introducing a nucleic acid vector comprising a SID-1 sequence, into at least some of the cells; and
(b) introducing a double-stranded RNA molecule having a sequence complementary to the target gene,
wherein the SID-1 sequence encodes a polypeptide having SID-1 activity.

34. A method for reducing the expression of a target gene in an animal, the method comprising:
(a) introducing a nucleic acid vector comprising a SID-1 sequence into the animal; and
(b) introducing a double-stranded RNA molecule having a sequence complementary to the target gene,
wherein the SID-1 sequence encodes a polypeptide having SID-1 activity.
